(19) United States
(12) Patent Application Publication
(10) Pub. No.: US 2012/0292534 A1
(43) Pub. Date: Nov. 22, 2012

(54) Multi-phase Gating for Radiation Treatment Delivery and Imaging

(76) Inventors: Sarah Geneser, Menlo Park, CA (US); Benjamin Pooya Fahimian, Menlo Park, CA (US); Lei Xing, Palo Alto, CA (US)

(21) Appl. No.: 13/475,231

(22) Filed: May 18, 2012

Related U.S. Application Data
(60) Provisional application No. 61/519,353, filed on May 20, 2011.

(51) Int. Cl. A61N 5/10; A61N 5/00

(52) U.S. Cl. 250/492.3; 250/492.1

(57) ABSTRACT

A multi-phase radiation therapy treatment method is provided that includes computational software to simultaneously optimize radiation plans for each phase of delivery. A specific realization of multi-phase therapy, dual gating, is described where the first radiation therapy treatment plan provides treatment during an inhale phase of a patient breathing cycle and the second radiation therapy treatment plan provides treatment during an exhale phase of the patient breathing cycle. Using a radiation therapy machine, the first radiation therapy treatment plan is delivered during the inhale phase and the second radiation therapy treatment plan is delivered during the exhale phase of the patient breathing cycle. An associated imaging method is provided for gated volumetric image guidance at multiple different phases in a single imaging acquisition.

### CROSS-REFERENCE TO RELATED APPLICATIONS

This application claims priority from U.S. Provisional Patent Application 61/519353 filed May 20, 2011, which is incorporated herein by reference.

### FIELD OF THE INVENTION

The present invention relates generally to radiotherapy. More particularly, the invention relates to treatment planning and delivery methods for dual-gated radiation therapy of moving targets.

### BACKGROUND OF THE INVENTION

Radiation therapy of moving targets, such as tumors located in or near abdominal and thoracic organs (e.g., lung, liver, pancreas, breast, and heart), is primarily complicated by respiratory-induced motion as well as cardiac and secondary patient motion. If not properly accounted for in treatment planning and delivery, such motion can result in underdosing the tumor target and overdosing nearby healthy tissues. The delivery component includes a gating signal to control the radiation source and associated systems (such as the collimator and imaging system) during treatment. The gating signal is any desired form of motion tracking monitoring system: which may include optical surrogates placed on the patient (such as the common RPM system), radiofrequency sources placed interstitially in the target or on the patient (such as Calypso), continual or frequent fiducial or target imaging, cardiac signal surrogates, or other physical detectors on the patient. Conventionally, a gating signal correlated to the breathing, cardiac, or tumor motion is used to trigger radiation delivery while the target is at a particular location. The core assumption of gated radiation therapy is that the target location corresponds to the surrogate signal during treatment planning and optimization and that this correspondence does not vary. Gated radiation therapy is delivered during time windows in which the tumor location is sufficiently reproducible and stationary in order to minimize dose delivery errors. However, restricting the beam-on time of the radiation source to a fraction of a breathing cycle prolongs radiation treatment times. Depending on patient motion patterns at the time of treatment, gating the treatment beam can significantly extend the amount of time required to complete a treatment delivery and increases treatment cost and patient discomfort. Longer delivery times also increase the chance that the tumor will deviate from its initial delivery setup position and result in delivery errors that reduce the likelihood of tumor control, increasing the risk of critical structure complications.

What is needed is a method of reducing radiation treatment times without restricting the beam-on time of the radiation source to a single fraction of a breathing cycle.

### SUMMARY OF THE INVENTION

To address the needs in the art, a dual-gated radiation therapy treatment method is provided that includes computational software to simultaneously optimize a first radiation therapy treatment plan and a second radiation therapy treatment plan, where the first radiation therapy treatment plan provides treatment during an inhale phase of a patient breathing cycle and the second radiation therapy treatment plan provides treatment during an exhale phase of the patient breathing cycle, and using a radiation therapy machine to alternately deliver the first radiation therapy treatment plan during the inhale phase and the second radiation therapy treatment plan during the exhale phase of the patient breathing cycle.

In one aspect of the invention, the first radiation therapy treatment plan includes inhale fluence weights wᵢ.

In another aspect of the invention, the second radiation therapy treatment plan comprises exhale fluence weights wₑ.

According to another aspect of the invention, the optimized accumulated dose includes a relation D_{DG}=Aₑwₑ+R(Aᵢwᵢ), where R is a mapping operator that registers an inhale CT volume Iᵢ to an exhale CT volume Iₑ.

In another aspect of the invention, the optimization of accumulated dose includes identifying optimal inhale radiation therapy treatment plan fluence weights wᵢ and optimal exhale radiation therapy treatment plan fluence weights wₑ to produce a dose distribution, where a desired minimum dose and a desired maximum dose are prescribed for all structures of interest. The desired dose for critical structures is zero.

According to a further aspect of the invention, the optimization comprises applying a registration to the inhale dose to the exhale anatomy at each step of the optimization.

In a further aspect of the invention, the registration provides a function that maps inhale geometry voxels to exhale geometry voxels, where the mapping is applied to the inhale dose matrix, Aᵢ, that computes the dose conferred by the inhale plan beamlets, wᵢ, to the inhale geometry in order to produce a dose matrix, Ãᵢ, that computes the inhale dose matrix directly on the exhale anatomy.

In yet another aspect of the invention, the radiation therapy type includes IMRT, arc, rapid-arc, VMAT, 3D, or conformal therapies.

According to one aspect of the invention, the radiation therapy treatment plan is a radiation source modality that includes photons, electrons, protons, or charged particles.

In a further aspect of the invention, a treatment gating window specification is based on an input that includes phase, amplitude, displacement, or alternative surrogate signals.

In a further aspect of the invention, the treatment gating window includes an adaptive treatment gating window.

In a further aspect of the invention, the treatment plans include using a multi-leaf collimator.

### BRIEF DESCRIPTION OF THE DRAWINGS

FIGS. 1a-1b show schematic drawings of dual-gated delivery, according to one embodiment of the invention, and conventional gating.

FIG. 2a shows the torso phantom used to generate dual-gated plans for various motion extents that was placed on a respiratory gating motion platform for 4DCT imaging, according to one embodiment of the invention.

FIG. 2b shows contoured structures are shown on the exhale CT in, according to one embodiment of the invention.

FIG. 3 shows the DG-IMRT dose distribution for a sagittal slice through the torso phantom, according to one embodiment of the invention.

FIGS. 4a-4f show the inhale and exhale dose components produced by the DG-IMRT planning framework for various motion extents, according to one embodiment of the invention.

FIGS. 5a-5c show the difference between the single- and dual-gated dose distributions in the presence of 1, 2, and 3 cm translation, respectively, according to one embodiment of the invention.

FIG. 6 shows DVHs for the single-gated and DG-IMRT treatment plans, according to one embodiment of the invention.

FIGS. 7a-7b show Dual-gated dose distributions according to the invention and single-gated exhale dose distributions.

FIG. 7c shows dose-volume histograms (DVH) for the dual and single gated plans.

FIGS. 8a-8c show volumetric image guidance in SBRT limited by respiratory motion artifacts, retrospective 4D-CBCT prospectively Gated CBCT as prospective solutions, and multi-phase gated CBCT according to the current invention.

FIG. 9 shows a schematic drawing of the multi-phase gating of the imager as the function of gantry rotation.

FIGS. 10a-10b show scanner reconstruction via a regular CBCT scanner reconstruction of the moving phantom.

FIGS. 11a-11b show scanner reconstruction via a prospective phase gated implementation, according to one embodiment of the current invention.

### DETAILED DESCRIPTION

In one embodiment of the invention, methods for image guidance of beam delivery for moving objects using prospectively gated multi-phase imaging are provided. Tomographic imaging of moving targets using on-board imaging modalities such as Cone-Beam CT (CBCT) has thus far been problematic because numerous projections at different angles are required for reconstruction, and patient motion during acquisition of the projections leads to severe motion artifacts in the reconstructed images. For accurate radiation treatment of thoracic and abdominal regions, an effective method for tomographic imaging moving targets during treatment is essential.

The current invention provides systems and methods that enable the prospective acquisition of tomographic images during multiple gating windows. The gating windows can be set to the respiratory or cardiac cycle phases of interest (e.g. corresponding to the phases for which the treatment plan was optimized or simulation tomographic images are available) prior to treatment. The projections are then physically acquired during their respective phase intervals using an imaging system triggered by the gating signal. The projections belonging to the same phase can be then reconstructed by tomographic reconstruction algorithms to obtain a static 3D tomographic image of the moving target at the multiple phases of interest. During patient positioning, such tomographic reconstructions can be compared to corresponding simulation images at particular phases for accurate 4D image guidance. Throughout this document, it is understood that motion of gating during a particular ‘phase’ of the gating cycle includes amplitude (a.k.a. displacement) threshold gating, gating derived based on a calculated mathematical phase of the gating trace, or in any way that the user desires to group characteristic portions of the gating trace.

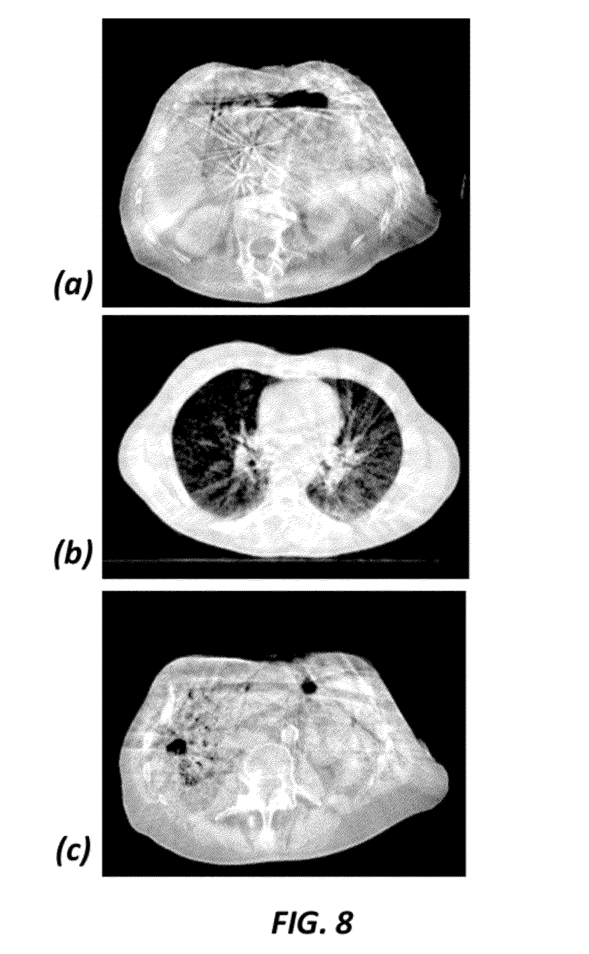

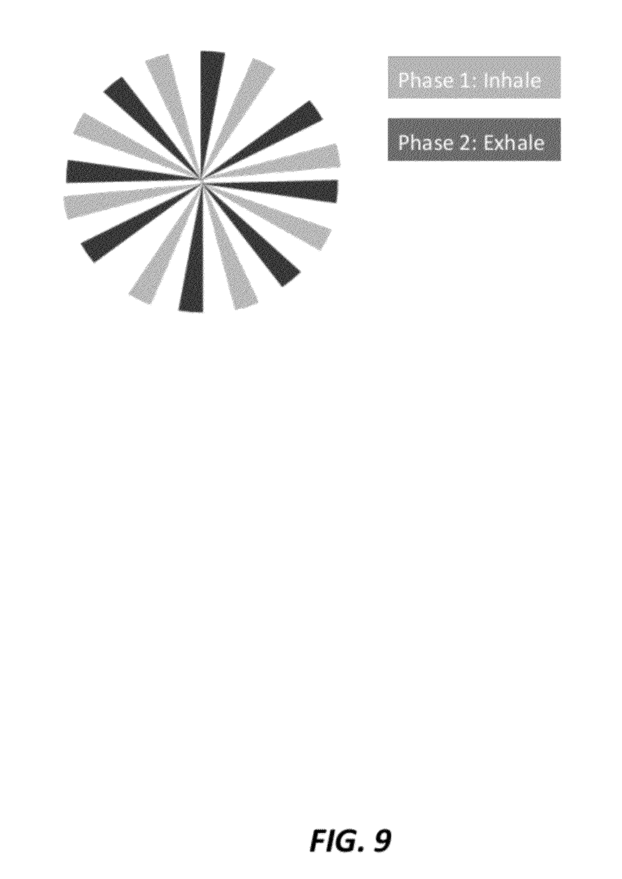

Currently, CBCT having on-board imagers connected to the delivery system are used for image guidance prior to the treatment. However, when the imaging object in the field of view is moving due to respiratory and cardiac motion, the image quality is considerably degraded due to violation of the underlying tomographic acquisition assumption that the projections are corresponding to the same object taken at different angles. Degradation of image quality is currently a significant limitation in tomographic image guidance. Such degradation of image quality is shown in FIGS. 8a-8c. In the literature, 4D CBCT methods have been proposed which acquire projections continuously across all phases, retrospectively bin projections into different phases, and reconstruct the object for particular phase. However, such a method is problematic as 1) it results in a high imaging dose delivered from the acquisition of many phases which are not entirely necessary for pre-treatment image guidance, and 2) it requires high computation time to reconstruct all phases, which is problematic because of the time-sensitive nature pre-treatment image guidance. A further embodiment provides modifying the tomographic imaging system by first enabling prospective gating and, secondly, uses multiple gating windows. This has been demonstrated and experimentally implemented by the inventors using the Varian TrueBeam LINAC equipped with kV on board imaging system. In a single scan, projections are acquired when the gating signal is within the prescribed phase or amplitude, such as those corresponding to inhale and exhale. Three embodiments are presented here for sources attached to gantry 1) either the gantry can stop and start rotation based on the gating signal, or 2) the gantry can roll back and forth to cover the same anatomical area at different gating phases, or 3) in a preferred embodiment, continuously rotate at a speed, which may be slower than the conventional non-gated acquisition, and have the beam come off and on the desired multiple phases based on the gating signal; embodiment 3 is shown in FIG. 9, which demonstrates the multi-phase gating of the imager as the function of gantry rotation.

While embodiment 1 and 2 ensures the most adequate sampling of projections, they result in slower acquisition time due to either physical inertial limitation either rolling the gantry back and forth as in embodiment 1, or stopping and restarting gantry motion in embodiment 2, In all embodiments, the projections acquired during the acquisition are then sorted according to their phase and reconstructed separately by existing tomographic algorithms. Such tomographic algorithms could include analytic methods, iterative methods, or compressed sensing methods for reconstruction of limited number of projections. In one embodiment, a faster gantry speed, and lower sparser number of projections can be acquired, and then reconstructed using compressed sensing or iterative algorithms more suited for sparse projection reconstruction. Relative to 4D retrospective acquisition method, the said method results in lowering of the dose as the images are only acquired about selected phases with the radiation beam is turned off during non-relevant phases, and lowering of reconstruction time as only selected phases are reconstructed.

Respiration-gated radiation therapy reduces normal tissue irradiation while maintaining tumor coverage by restricting beam delivery to a portion of the respiration cycle. However, this restriction prolongs treatment. To improve delivery efficiency while preserving the dosimetric advantage of gating, a dual-gated intensity modulated radiation therapy (DG-IMRT) method, which is a subset of multi-phase gating, is provided that delivers radiation at both inhale and exhale and an inverse treatment planning algorithm is developed for the method.

Instead of producing and delivering a treatment plan for a single specific respiratory phase, one embodiment of the current invention provides delivery over multiple temporal periods (henceforth referred to equivalently as ‘multi-phase’), such as both inhale and exhale phases. In addition to the proposed multi-phase delivery, novel treatment planning optimization algorithms necessary for such treatments are disclosed.

Until now, gating at multiple phases has not been achieved because the effect of a treatment plan, and hence the apertures of the radiation beam, is only known for a single phase, and hence gating besides the plan phase result in incorrect dosing of the target and organs. This invention overcomes these issues with novel treatment algorithms that model and account for the multi-phase a system that enables gating during multiple phase periods.

Radiation therapy treatment planning involves identifying a set of machine delivery parameters that result in deposited dose to the tissues that match the physician prescribed dose within clinical tolerances. This is accomplished by medical physicists using dedicated treatment planning software that 1) models the radiation dose deposited to the patient in response to specific machine parameters and 2) optimizes the machine parameters to produce a clinically acceptable patient dose. Radiation therapy treatment planning systems previously did not explicitly include multi-phase planning methods.

In one embodiment of the invention, software is leveraged to provide multi-phase treatments by first generating clinical plans for each phase individually. The individual plans are then mapped to a reference phase using deformable image registration methods and summed using dose summing tools to determine the total dose deposited to the tumor and surrounding tissues. In the event that this alone does not produce clinically acceptable plans, each individual plan can then be normalized to produce a plan within clinical tolerances. In the case of dual-phase plans, the normalization process will not require optimization, but in the case of three or more plans, it is likely that the normalization parameters will require optimization to produce a clinically relevant plan.

In contrast to conventional gated delivery, for which beamlet weights are optimized over patient anatomy obtained from a CT volume corresponding to a single respiratory phase, one embodiment of the invention provides a novel treatment planning method that optimizes over images from multiple phases (e.g. CT volumes corresponding to inhale and exhale). These distinct beam fluences (wᵢ and wₑ) correspond to a separate leaf sequence for each delivery phase and are designed to be delivered alternately at the two different respiratory phases.

Once an acceptable multi-phase plan has been developed, the plans are delivered by phase-tagging each of the plans to a specific gating window and then instructing the machine to deliver that plan when the external surrogate signal falls within the gating window corresponding to each plan. In another embodiment, in the case of dual-phase gated therapy, there are two separate plans and the MLC's would initially move into position for the first plan, and wait to beam on until the surrogate is within the first gating window. Once the surrogate exits the first gating window, the MLC's then move into position for the second plan, and wait to beam on until the surrogate falls within the second gating window. When the surrogate exits the second gating window, the MLC's move into position for the first plan and the process repeats until the entire plan has been delivered.

Some advantages of the current invention include a reduction in treatment times due to increased linear accelerator beam-on time, leveraging of four-dimensional information (4DCT) to produce a treatment plan for which tumor margins can be reduced owing to increased geometric tumor location certainty of delivery, having an algorithm with a general form, and can be used in almost all of the current radiation treatment modalities, and the optimization for treatment planning is efficient both in that it only slightly increases the computational effort required to obtain a good solution and in that it precludes the need for medical physicists to design two separate plans for each phase (inhale and exhale).

In DG-IMRT, the dose is delivered alternately during inhale and exhale gating windows. The inverse planning framework of the current invention identifies the DG-IMRT inhale and exhale fluence maps that produce the optimal accumulated dose distribution. Accumulated DG-IMRT doses are computed by mapping the inhale dose to the exhale anatomy using deformable image registration and summing the exhale and registered inhale dose distributions.

In one embodiment an algorithm is then provided that simultaneously optimizes both the inhale and exhale fluence maps. Subsequent to the mathematical development, the performance of DG-IMRT treatment planning is demonstrated on a phantom case undergoing 1, 2, and 3 cm periodic translation and a lung patient case with approximately 1.5 cm tumor motion. For both the phantom and patient cases, the quality of the dual-gated plans are found to be comparable to the conventional plan gated at exhale alone.

Respiratory motion presents considerable challenges for the treatment of thoracic and abdominal tumors. Respiratory-gated radiotherapy is a widely-employed means of treating tumors that undergo large motion during breathing. This approach effectively simplifies a four-dimensional (4D) treatment planning calculation to a three-dimensional (3D) one by restricting delivery to a portion of the respiratory cycle. The resulting reduction in tumor motion during delivery enables tumor coverage with a smaller planned target volume (PTV) which, in turn, improves normal tissue sparing. However, gating reduces the duty cycle and can appreciably extend delivery.

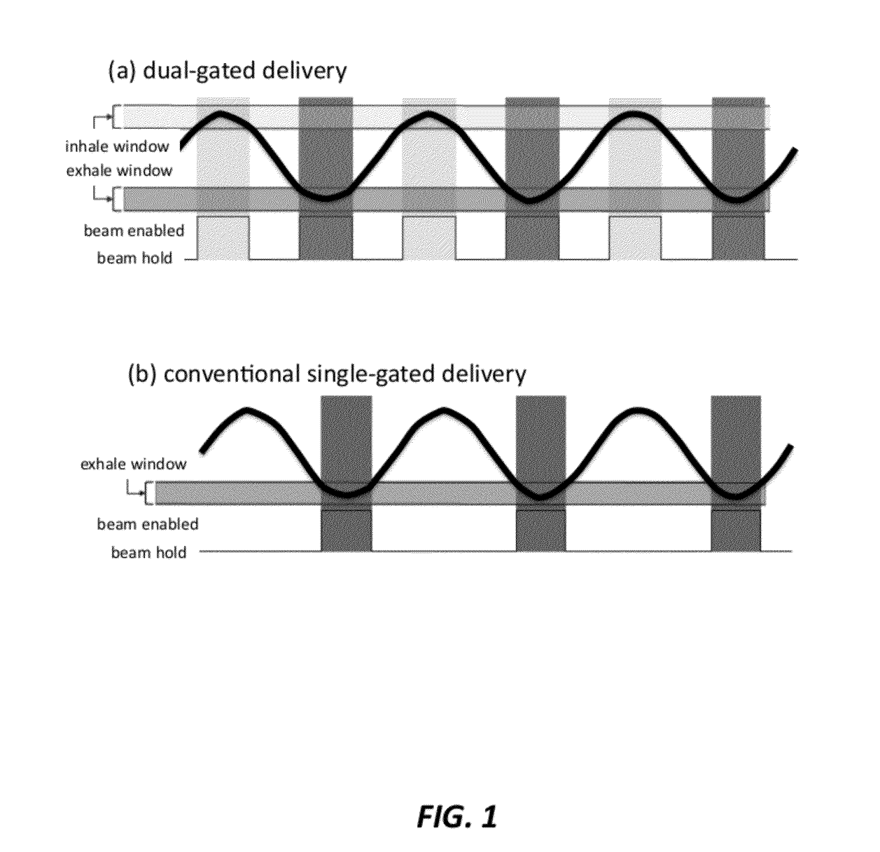

Prolonged treatment time increases the likelihood of several potential deleterious effects, including; deviations between the planned and delivered dose distributions resulting from postural shifts during delivery, decreased clinical work-flow, increased patient discomfort, and decreased biological dose effectiveness. Improving gated delivery efficiency is particularly important for stereotactic body radiation therapy (SBRT) and stereotactic ablative radiation therapy (SABR), because of the already protracted delivery associated with large dose fractions. To improve tumor control and normal tissue sparing for tumors affected by respiratory motion, it is clinically worthwhile to develop efficient radiation treatment delivery methods. A dual-gated intensity modulated radiation therapy (DG-IMRT) technique is provided for delivering radiation at both inhale and exhale (rather than a single gating window) to take advantage of the natural pauses that occur at peak-inspiration and end-exhalation. A schematic illustration of dual-gated delivery is shown alongside conventional gating in FIGS. 1a-1b. To a large extent, DG-IMRT provides the advantages of 4D radiation therapy (4DRT), e.g., improving delivery efficiency without compromising dose conformality, while avoiding the technical barriers associated with MLC and couch tracking. Present below is an example a treatment planning method for generating DG-IMRT plans.

DG-IMRT includes two individual IMRT plans to be alternately delivered during inhale and exhale gating windows as shown in FIG. 1a. The aim of DG-IMRT planning is to find the inhale and exhale fluence maps that produce the optimal cumulative dose distribution.

As opposed to optimizing inhale and exhale plans as a decoupled system, an inverse planning technique is provided to optimize simultaneously over the accumulated dose distribution.

The details of dose calculation, treatment planning, and evaluation are described here. The inhale and exhale fluence maps are partitioned into a collection of individual beamlets, with Nᵤ y-axis divisions corresponding to the multileaf collimator (MLC) geometry and Nᵥ x-axis leaf motion steps. Patient geometry and tissue electron density are derived from the inhale and exhale CT volumes, Iᵢ and Iₑ, respectively. Dose deposition is formulated as a linear system d=Aw, where w is the vector composed of the (N_{y}×Nₓ) IMRT beamlet intensities for each of the N_{f} treatment fields, and A is the dose matrix composed of elements Aij that describe the dose contribution of the the j^{th} beamlet of unit intensity to the dose at the i^{th} voxel. Dose matrices used in this example were calculated with the VMC++ simulation implemented in Computational Environment for Radiotherapy Research (CERR).

To simultaneously optimize the inhale and exhale fluences, the accumulated dose resulting from the two fluences (wᵢ and wₑ) must be computed. The dose matrices corresponding to patient anatomy at inhale and exhale are denoted Aᵢ and Aₑ, respectively. The accumulated dose is written as

d_{DG}=Aₑwₑ+R(Aᵢwᵢ), (1)

where R is a mapping operator that registers the inhale CT volume, Iᵢ to the exhale CT volume, Iₑ. For this example, the implemented inverse treatment planning system uses the medical image registration method with elastic regularization constraints available in the deformable image registration and adaptive radiotherapy (DIRART) MATLAB toolbox as part of CERR.

DG-IMRT treatment planning simultaneously optimizes the accumulated dose to identify optimal inhale and exhale IMRT beamlet weights, wᵢ and wₑ, to produce a dose distribution that most closely matches the prescribed dose to the tumor target while limiting the dose to critical structures. The problem is mathematically defined as

$${minimize}_{{w}_{i} , {w}_{e}} {\sum }_{s} {λ}_{s} ‖ {[ {A}_{e} {w}_{e} ]}_{s} + {[ R ( {A}_{i} {w}_{i} ) ]}_{s} - {D}_{s} ‖ + β {\sum }_{p ∈ i , e} {\sum }_{f = 1}^{{N}_{f}} {\sum }_{u = 2}^{{N}_{u}} {\sum }_{v = 2}^{{N}_{v}} ( \vert {w}_{u , v , f , p} - {w}_{u - 1 , v , f , p} \vert + \vert {w}_{u , v , f , p} - {w}_{u , v - 1 , f , p} \vert ) \\ subject to 0 \leq {w}_{i} \leq {w}_{max} \\ 0 \leq {w}_{e} \leq {w}_{max} \\ {D}_{{min}_{s}} \leq ( {[ {A}_{e} {w}_{e} ]}_{s} + {[ R ( {A}_{i} {w}_{e} ) ]}_{s} ) \leq {D}_{{max}_{s}} , \qquad ( 2 )$$

where desired, minimum, and maximum doses (Dₛ, Dminₛ and Dmaxₛ) are prescribed for each structure of interest, s, and [•], denotes the vector of dose values at voxels within s. For critical structures, Dminₛ is zero. The beamlet intensities are non-negative and limited by wₘₐₓ, resulting in a constrained optimization problem. A relative importance weight, rₛ, is defined for each of the structures and

$${λ}_{s} = \frac{{r}_{s}}{\sqrt{{M}_{s}}} .$$

A total variation regularization term, weighted by β is included in the objective function to encourage piecewise continuous fluence maps that require fewer MLC sequences to deliver. The resulting inverse planning system is optimized using MOSEK's optimization routine.

Equation 2, requires applying the registration, R, to the inhale dose at each step of the optimization. To facilitate computation, this deformation is incorporated into the inhale dose matrix to enable direct computation of the inhale dose mapped to the exhale anatomy, R(dᵢ)=R(Aᵢwᵢ)=Ãᵢwᵢ.

The inhale dose at the j^{th} voxel within the inhale geometry is [dᵢ]ⱼ=[Aᵢ]ⱼwᵢ where [Aᵢ]ⱼ is the j^{th} row of the inhale dose matrix. The registration R provides a function, f, that maps a subset of voxels, j, to a particular voxel, k in the exhale geometry. This mapping can be applied to the inhale dose matrix, so that for each voxel, k, in the exhale geometry, the inhale dose at that voxel is [f ([Aᵢ]ⱼ)]ₖwᵢ=[(Ãᵢ)ₖwᵢ, where the k^{th} row of the mapped inhale matrix, Ãᵢ, is composed of the function f applied to the j rows of Aᵢ. The accumulated dose distribution on the exhale anatomy is d_{DG}=Ãᵢwᵢ+Aₑwₑ, avoiding repeatedly mapping the inhale dose distribution onto the exhale geometry.

The DG-IMRT planning method of the current invention was evaluated using a Quasar™ Multi-Purpose Body Phantom (Modus Medical Devices Inc., London, Ontario) undergoing 1, 2, and 3 cm of translational motion in the superior/inferior (SI) direction. For each motion extent, a DG-IMRT plan was created using the algorithm described above and the resulting dose distributions were assessed. For comparison, a conventional plan optimized on the exhale CT was also generated.

The Quasar phantom is composed of multiple inserts to mimic different organ tissue properties within the torso. The inserts include hypothetical lungs, heart, spinal cord, and tumor. 4DCTs were acquired using the Real-Time Position Management (RPM) System with the phantom on top of a Respiratory Gating Platform, undergoing 4 s periodic motion in the SI axis. The hypothetical organ inserts were segmented on both inhale and exhale CT volumes and used for the DG-IMRT treatment planning presented herein.

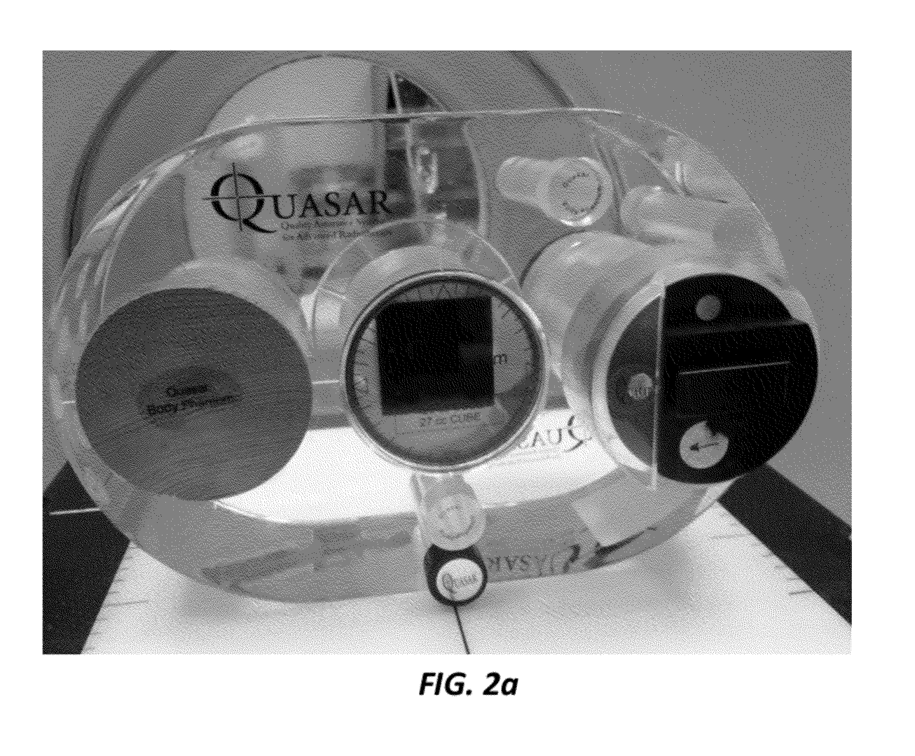

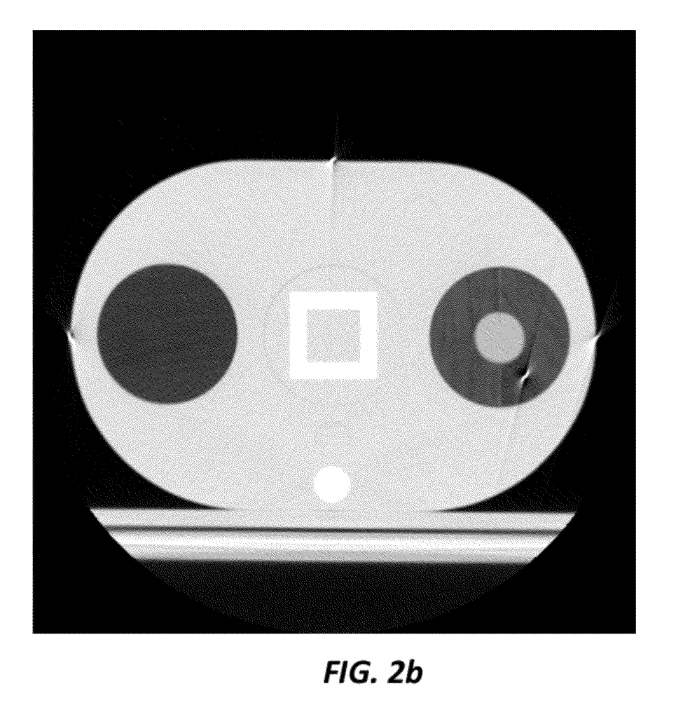

FIG. 2a shows the torso phantom used to generate dual-gated plans for various motion extents that was placed on a respiratory gating motion platform for 4DCT imaging. Contoured structures are shown on the exhale CT in FIG. 2b.

In a patient study, the dual-gated treatment optimization method of the current invention was retrospectively applied to a lung cancer patient case with approximately 1.5 cm tumor motion in the SI direction.

The 4DCT images and anatomical contours (e.g., PTV, lung, heart, etc.) used in the study were obtained from the original treatment plan. For comparison, exhale was separately planned. The dose distributions of the two plans were compared quantitatively.

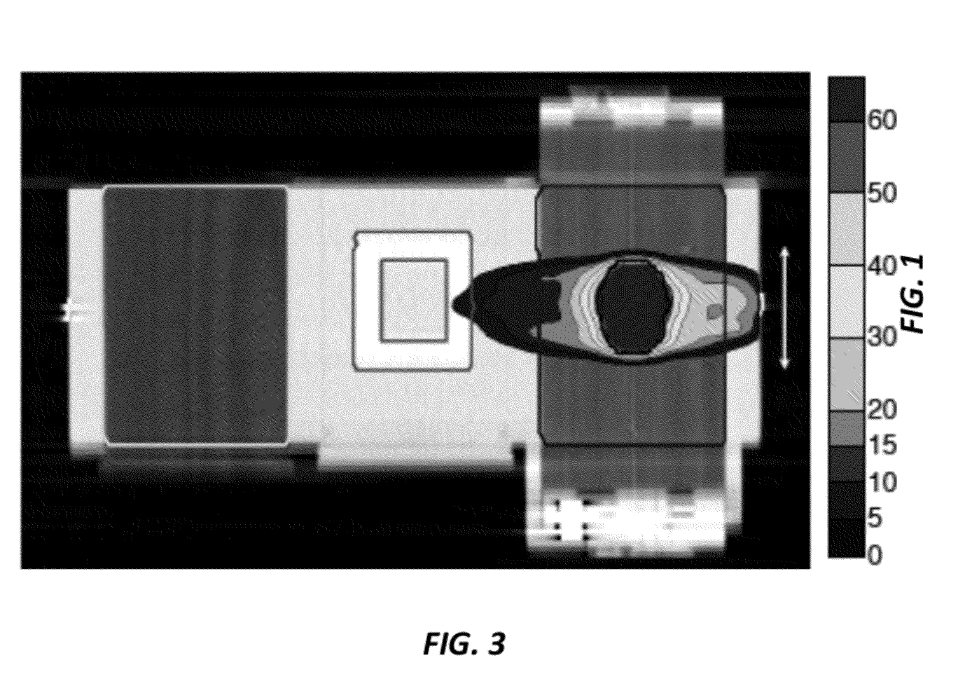

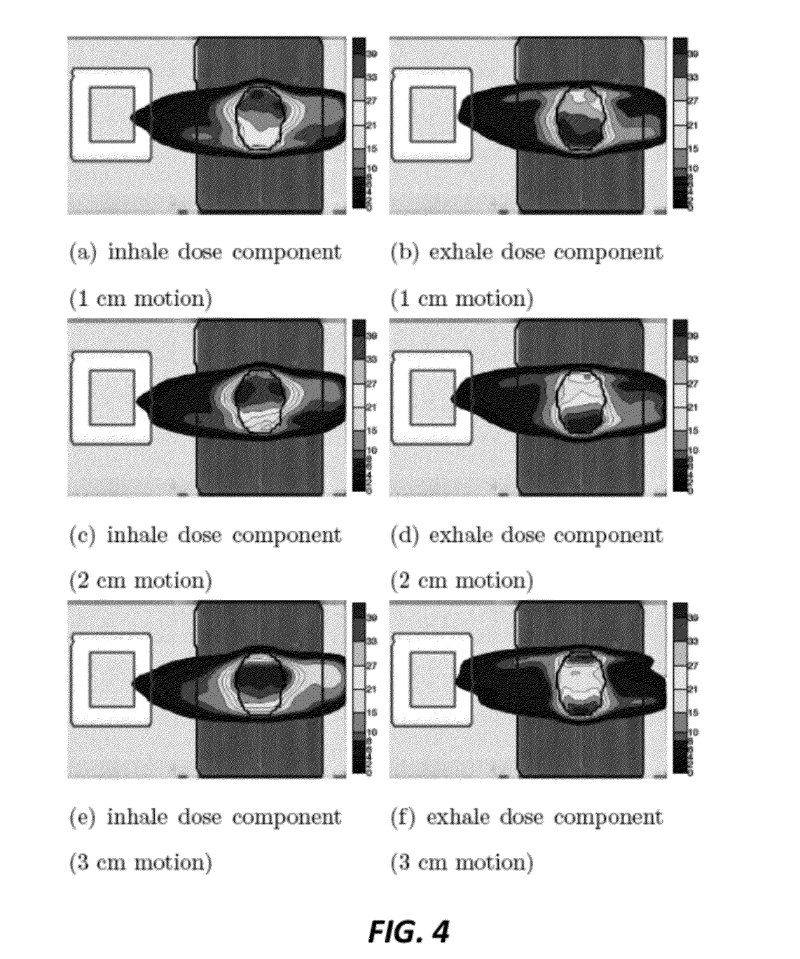

In a four-dimensional phantom study, FIG. 3 shows the DG-IMRT dose distribution for a sagittal slice through the torso phantom. The arrow indicates the direction of SI transitional motion. The contours correspond to the structures in FIG. 2a, the arrow indicates the direction of SI translational motion, and the color-wash indicates the dose levels (in Gy) for the plan. The inhale and exhale dose components produced by the DG-IMRT planning framework are shown in FIGS. 4a-4f for various motion extents. Note that taken individually, the inhale and exhale IMRT plans are not homogeneous within, nor conformal to, the PTV. However, the accumulated inhale and exhale dose distribution meets the planning objectives. Overall, the difference between the dual-gated and conventionally gated plans are within 5% of the prescribed dose of 60 Gy.

The main benefit of DG-IMRT lies in the ability to deliver dose during both inhale and exhale windows. For the phantom case with sinusoidal translation, the planned DG-IMRT delivery doubles the duty cycle as compared to conventional gating at exhale.

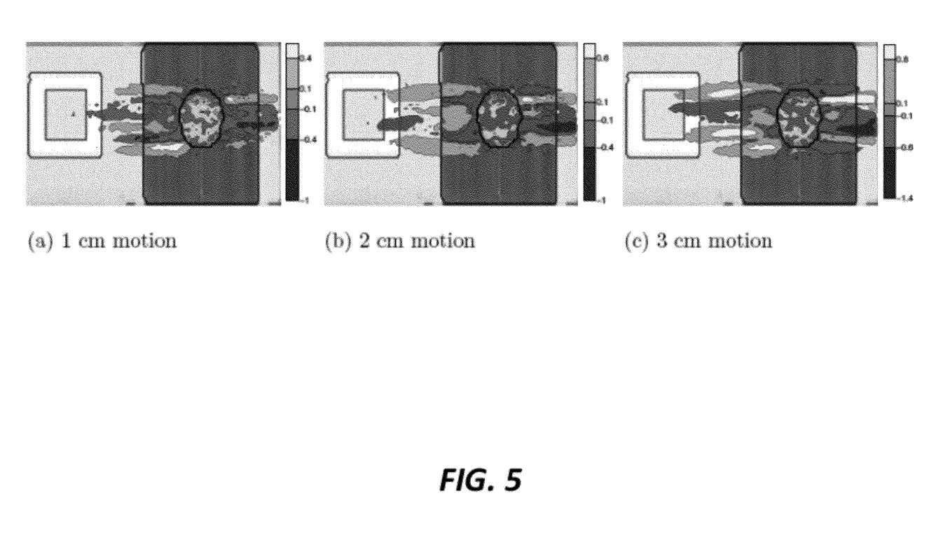

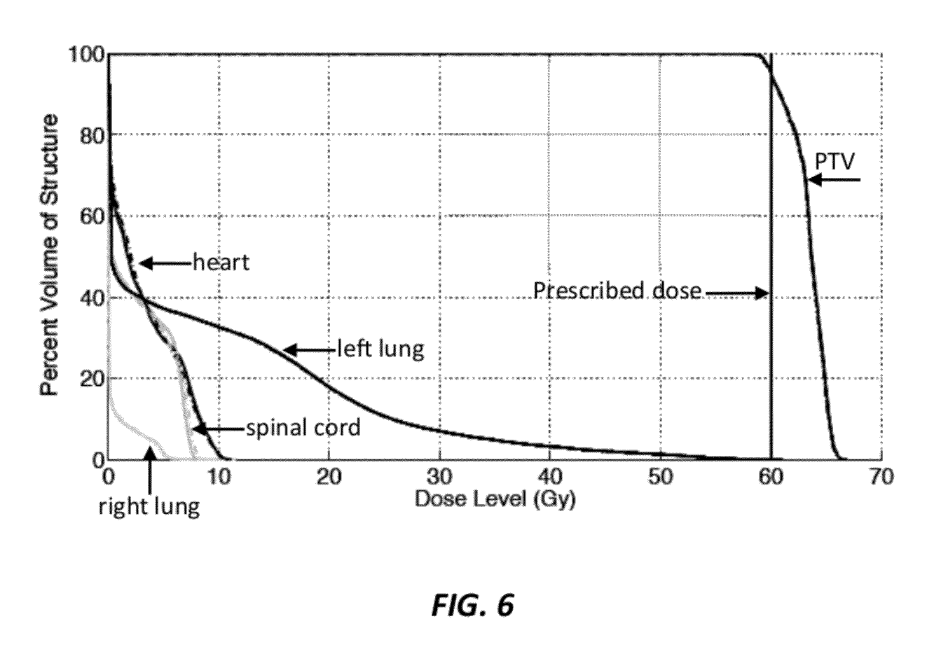

FIGS. 5a-5c show the difference between the single- and dual-gated dose distributions in the presence of 1, 2, and 3 cm translation, respectively. The discrepancy in dose increases slightly as the motion extent increases. The differences between the dose for the single-gated and DG-IMRT plans exhibit a slight banding pattern as a result of the variation in fluence modulation. DVHs for the single-gated and DG-IMRT treatment plans are presented in FIG. 6. The PTV and ipsilateral lung DVHs are almost identical for all plans, demonstrating that DG-IMRT is capable of producing sensible treatment plans. The solid, dashed, dotted, and dash-dotted lines correspond to the single-gated and dual-gated 1 cm, 2 cm, and 3 cm plans, respectively. The DVHs for each of the plans are nearly indistinguishable.

Overall, dose metrics for the DG-IMRT plans (Table 1) indicate that the single- and dual gated plans are very similar despite increasing target motion from 1 to 3 cm. The difference between single-gated and DG-IMRT PTV minimum and maximum doses are small for all motion extents. For example, the maximal dose to the heart and spinal cord is slightly larger for the DG-IMRT plan (increased by up to 0.2 Gy (1.8%) for the 3 cm plan and 0.4 Gy (4.8%) for the 1 and 2 cm plans, respectively). DG-IMRT conformity index differs from that of the exhale-gated plan by less than 0.1%.

TABLE I
Dosimetric comparison of dual- and single-gated phantom plans

| | Single-Gated | DG-IMRT | | |
|---|---|---|---|---|
| | Plan at Exhale | (1 cm) | (2 cm) | (3 cm) |
| min dose_{PTV} (Gy) | 57.7 | 57.6 | 57.6 | 57.5 |
| max dose_{PTV} (Gy) | 66.9 | 66.7 | 66.6 | 66.7 |
| max dose_{left lung} (Gy) | 61.0 | 61.0 | 60.7 | 60.7 |
| max dose_{right lung} (Gy) | 11.6 | 10.9 | 10.3 | 9.1 |
| max doseₕₑₐᵣₜ (Gy) | 11.1 | 11.1 | 11.0 | 11.3 |
| max dose_{spinal cord} (Gy) | 8.3 | 8.1 | 8.1 | 8.7 |

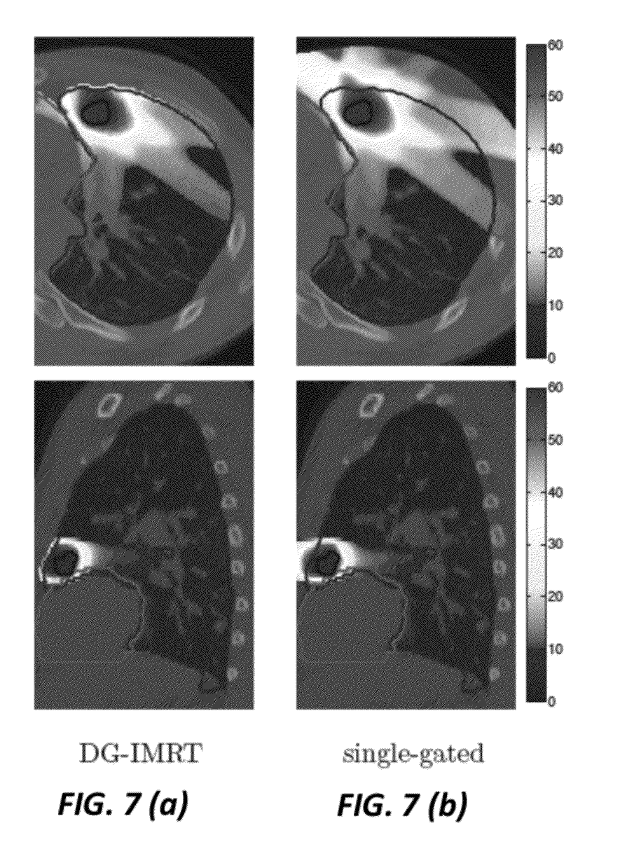

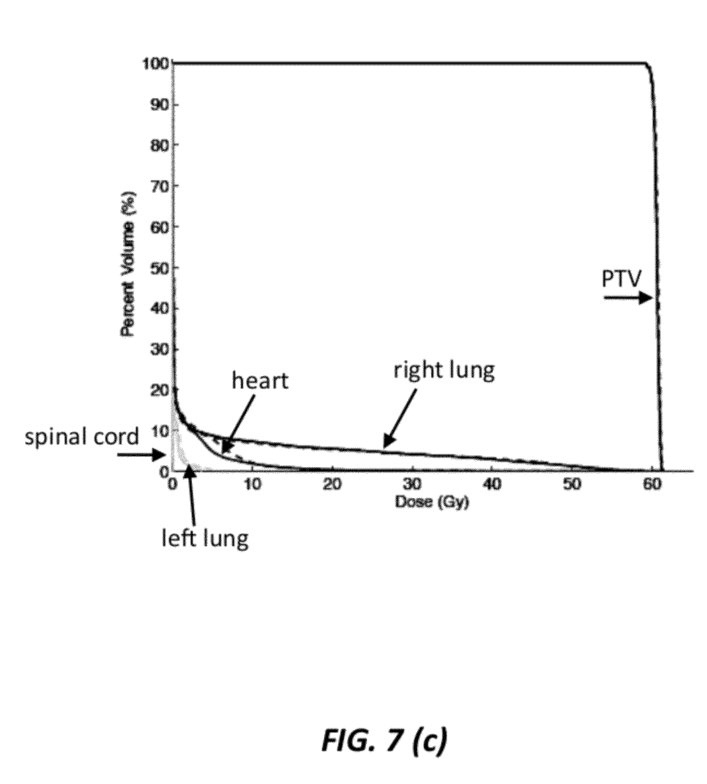

Dual- and single-gated exhale dose distributions and DVHs are shown in FIGS. 7a-7b, respectively, and the dose statistics for the two plans are summarized in (Table II). The single-gated plan was optimized for the case, and then importance weights were assigned so that the resulting DG-IMRT plan provided similar dose characteristics to the single-gated plan. The DVHs of the dual-and single-gated exhale plans are indicated by solid and dashed lines, respectively.

TABLE II
Dosimetric comparison of the dual- and single-gated plans for
the patient case

| | | Exhale-Gated |
|---|---|---|
| | DG-IMRT Plan | Plan |
| PTV dose (min, mean, max) (Gy) | (59.3, 60.6, 61.2) | (59.3, 60.7, 61.5) |
| left lung dose (mean, max) (Gy) | (2.3, 61.0) | (2.3, 61.2) |
| right lung dose (mean, max) (Gy) | (0.2, 3.7) | (0.2, 5.4) |
| heart dose (mean, max) (Gy) | (0.8, 56.1) | (1.0, 55.2) |
| spinal cord dose (mean, max) (Gy) | (0.003, 0.04) | (0.004, 0.1) |
| conformity index | 0.8697 | 0.8516 |

These results demonstrate the ability of the proposed DG-IMRT planning method to produce dose distributions that meet clinical IMRT dose requirements.

DG-IMRT leverages the natural respiratory pauses at peak-of-inhale and end-of-exhale, which has comparatively stable and reproducible portions of the respiratory cycle. In doing so, DG-IMRT improves treatment efficiency. Relative to tracking techniques, DG-IMRT is more accurate because it avoids irradiation during more unpredictable portions of respiration. The strategy may also prove more clinically feasible because it obviates the need for real-time MLC- or couch-based tracking.

DG-IMRT treatment enhancement under a free breathing scenario without coaching guidance and/or intervention is proportional to the window durations around peak inhale and end exhale over the full respiratory cycle duration. Analysis of free breathing behavior has demonstrated that patient's typically spend more time in the exhale window than the inhale window in the absence of coaching. In the presence of free-breathing, a nearly two-fold improvement in DG-IMRT delivery efficiency enhancement is expected. With instructed breathing and/or visual and audio coaching, a short breath-hold at both inhale and exhale can be utilized by most patients to increase the proportion of time spent at both peak inhalation and end exhalation in order to improve DG-IMRT-enabled delivery efficiency gain beyond double.

Practical implementation of DG-IMRT necessitates the ability to gate the radiation source at two distinct periods per respiratory cycle, and the ability for the MLCs to conform to the optimized apertures corresponding to each phase. While gating on a single phase is currently implementable on most modern LINACs, gating on both inhale and exhale is yet to be clinically developed. Proof of principle implementation is demonstrated using the Varian TrueBeam™ STx in conjunction with Developer Mode scripting. In particular, the TrueBeam™ STx is controlled via custom XML scripts, through which Boolean operators enable beam delivery on both inhale and exhale, and the MLC apertures are specified leaf by leaf as function of monitor unit control points.

In summary, DG-IMRT dual-gating has been established and an inverse planning framework for the new gating scheme has been provided. As compared to the existing respiratory-gating technique, a major advantage of dual-gating DG-IMRT is that it substantially reduces treatment duration with a modest but practically achievable increase in complexity of the treatment planning and delivery processes. Simultaneous optimization speeds up the treatment planning workflow and may provide dosimetric advantages over optimizing inhale and exhale separately. Moreover, irradiating during two respiratory windows provides an additional degree of freedom for dose spreading and normal tissue toxicity reduction. With as much as two times faster delivery, dual-gating DG-IMRT provides a clinically feasible means of increasing treatment throughput.

In a further example of one embodiment of the current invention, a prospective respiratory gated CBCT on a clinical LINAC based OBI system was implemented and evaluated using the Varian TrueBeam™ STx's triggered imaging capabilities in conjunction with its Developer Mode functionality. XML scripts, allowing for triggered acquisition of radiographic projections during specified gating windows were developed in accordance to the Varian Developer Mode schema. A motion stage, coupled with an image quality phantom, was used to simulate patient motion. Optical tracking of a reflector block attached to the phantom was used as the gating signal from which the image acquisition triggered by. The gated projections were acquired at 100 kVp, 40 mA, and 10 ms setting with no bowtie filter, for a variety of different gating windows and respiratory trajectories and reconstructed using an in-house FDK reconstruction algorithm. The image quality was quantified against conventional non-gated CBCT acquisition as well as the ideal image derived from a high quality scan of the phantom under static conditions.

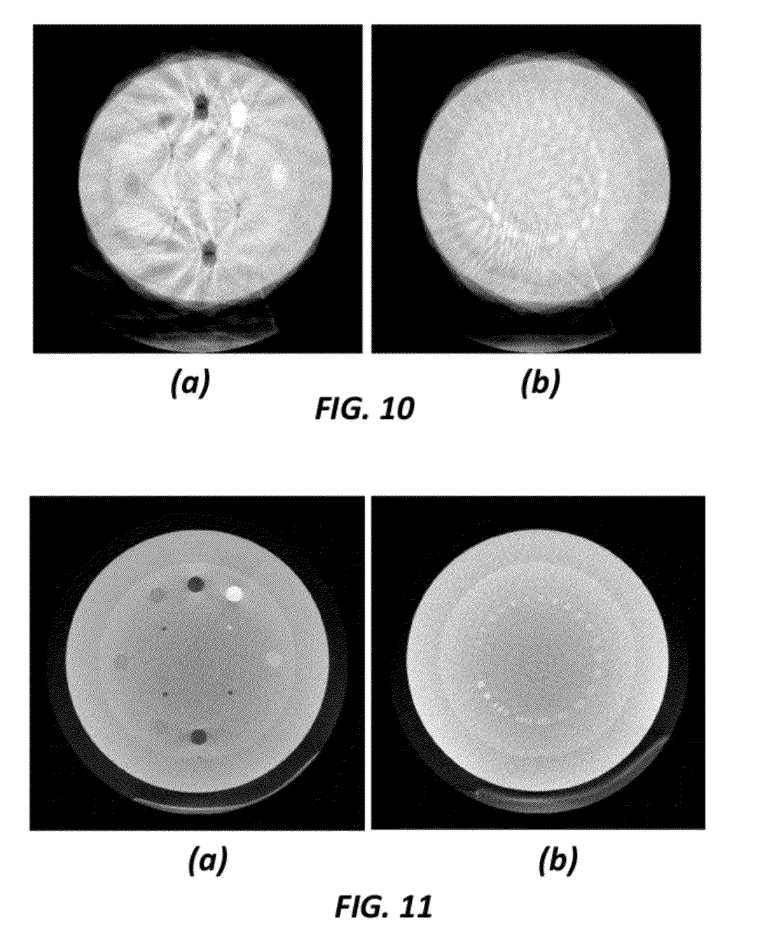

For an elliptical trajectory of the image quality phantom, with a 2 cm SI/1 cm AP displacement and a 4 second cycle period, the resulting scanner reconstruction via a regular non-gated CBCT, and the prospective phase gated implementation is given in FIGS. 10a-10b and FIGS. 11a-11b,

respectively. For the prospectively gated implementation, the gating was set to 30-70% of the detected period of the surrogate placed on the phantom, which for a 360° rotation resulted in a total of 1696 projections being collected. Comparisons of the reconstructions show significant elimination of motion artifacts resulting in the improvement of image quality and geometric fidelity in the prospective gated implementation. Quantitatively, resolution bars up to 6 lp/cm are visible in the gated implementation, while for the regular CBCT, the spacing and the geometrical structure of the resolution bars is not readily discernable. The acquisition confirmed the functionality of the imaging in Developer Mode, and the image quality of the gated CBCT confirmed the stability and accuracy of the overall triggered imaging system.

FIGS. 8a-8c show volumetric image guidance in SBRT, where shown in FIG. 8a, volumetric image guidance for SBRT is limited by respiratory motion artifacts due to inconsistency in acquired phase of projections. FIG. 8b shows retrospective 4D-CBCT prospectively Gated CBCT as prospective solutions, and FIG. 8c shows multi-phase gated CBCT implemented using custom scripting in TrueBeam Developer Mode Prospectively Gated CBCT.

The present invention has now been described in accordance with several exemplary embodiments, which are intended to be illustrative in all aspects, rather than restrictive. Thus, the present invention is capable of many variations in detailed implementation, which may be derived from the description contained herein by a person of ordinary skill in the art. For example Dual/multi-phase gating can be used for all treatment techniques IMRT, arc, rapid-arc, VMAT, 3D, conformal therapies. There is no restriction put on the radiation source or delivery system. The radiation source modality may be photons, electrons, protons, charged particles, etc. The gating window specification may be based on phase, amplitude or displacement, alternative surrogate signals, or a combination of surrogates. The gating windows may be adaptive. Dual/multi-phase gating plans can be produced individually using conventional techniques, summed to determine the total effect on dose deposition, and normalized individually to produce a clinically acceptable plan. Dual/multi-phase gating plans can be produced by instead solving over a single set of beamlet fluences (rather than a separate set for each respiratory phase). This reduces the degrees of freedom of the optimization problem by half and may prevent achieving clinically viable plans in some cases.

All such variations are considered to be within the scope and spirit of the present invention as defined by the following claims and their legal equivalents.

What is claimed:

1. A dual-gated radiation therapy treatment method, comprising:
a. using a computer to operate computational software to simultaneously optimize a first radiation therapy treatment plan and a second radiation therapy treatment plan, wherein said first radiation therapy treatment plan provides treatment during an inhale phase of a patient breathing cycle and said second radiation therapy treatment plan provides treatment during an exhale phase of said patient breathing cycle; and
b. using a radiation therapy machine to alternately deliver said first radiation therapy treatment plan during said inhale phase and said second radiation therapy treatment plan during said exhale phase of said patient breathing cycle.

2. The method according to claim 1, wherein said first radiation therapy treatment plan comprises inhale fluence weights wᵢ.

3. The method according to claim 1, wherein said second radiation therapy treatment plan comprises exhale fluence weights wₑ.

4. The method according to claim 1, wherein said optimized accumulated dose comprises a relation d_{DG}=Aₑwₑ+R(Aᵢwᵢ), wherein R is a mapping operator that registers an inhale CT volume Iᵢ to an exhale CT volume Iₑ.

5. The method according to claim 1, wherein said optimization of accumulated dose comprises identifying optimal inhale radiation therapy treatment plan fluence weights wᵢ and optimal exhale radiation therapy treatment plan fluence weights wₑ to produce a dose distribution, wherein a desired minimum dose and a desired maximum dose are prescribed for all structures of interest.

6. The method according to claim 5, wherein said desired minimum dose is zero for critical structures.

7. The method according to claim 1, wherein said optimization comprises applying a registration to an inhale dose at each step of said optimization, wherein direct computation of said inhale dose is mapped to an exhale anatomy.

8. The method according to claim 1, wherein said registration provides a function that maps inhale geometry voxels to exhale geometry voxels, wherein said mapping is applied to an inhale dose matrix Aᵢ that computes the dose conferred by the inhale plan wᵢ to an inhale geometry.

9. The method according to claim 1, wherein said radiation therapy type is selected from the group consisting of IMRT, arc, rapid-arc, VMAT, 3D, and conformal therapies.

10. The method according to claim 1, wherein said radiation therapy treatment plan comprises a radiation source modality selected from the group consisting of photons, electrons, protons, and charged particles.

11. The method according to claim 1, wherein a treatment gating window specification is based on an input selected from the group consisting of phase, amplitude, displacement, and alternative surrogate signals.

12. The method according to claim 1, wherein said treatment gating window comprises an adaptive treatment gating window.

13. The method according to claim 1, wherein said treatment plans comprise using a multi-leaf collimator.
